(19) United States
(12) Patent Application Publication
(10) Pub. No.: US 2006/0162005 A1
(43) Pub. Date: Jul. 20, 2006

(54) Methods for producing transgenic medaka

(76) Inventor: Huai-Jen Tsai, Taipei (TW)

Correspondence Address: HARNESS, DICKEY & PIERCE, P.L.C., P.O. BOX 8910, RESTON, VA, 20195, US

(21) Appl. No.: 11/377,838

(22) Filed: Mar. 17, 2006

Related U.S. Application Data
(62) Division of application No. 10/752,687, filed on Jan. 8, 2004.

(51) Int. Cl. A01K 67/027

(52) U.S. Cl. 800/20

(57) ABSTRACT

The invention relates to a recombinant DNA method for producing transgenic medaka. The invention also relates to novel gene fragments for producing the transgenic medaka. The invention further relates to novel transgenic medaka.

### CROSS REFERENCE TO RELATED APPLICATION

This is a divisional application of U.S. application Ser. No. 10/752,687 filed on Jan. 8, 2004, the entire contents of which is hereby incorporated by reference.

### FIELD OF THE INVENTION

The invention relates to a method to produce a novel transgenic medaka. The invention also relates to novel nuclei acid fragments and novel transgenic medaka.

### BACKGROUND OF THE INVENTION

Ornamental fish is part of the fishery business and has a global market. Therefore, using recombinant DNA and transgenic techniques to modify the phenotypes of ornamental fish has great market value.

Transgenic fish studies make use of genes that are driven by both heterologous and homologous sources of regulatory element, and originate from constitutive or tissue-specific expression genes. Control elements include genes from antifreeze protein, mouse metallothionein, chicken δ-crystalline, carp β-actin, salmon histone H3 and carp α-globin and so on. However, there are important drawbacks to the use of these DNA elements in transgenic fish, including low expression efficiency and the mosaic expression of transgene patterns.

The microinjection into mekada eggs of lacZ reporter gene driven by the mekada β-actin promoter results in the transient expression of the lacZ gene, even in the F1 generation, though expression is low and highly mosaic. Hamada et al. reported a similar result in medaka embryos derived from eggs microinjected with green fluorescence protein fused with the medaka β-actin promoter (Hamada et al., 1998, Mol Marine Biol Biotechnol 7: 173-180).

Unfortunately, conventional transgenic technologies can only produce transgenic fish emitting mosaic or weak fluorescence. The fluorescence of these transgenic fish could only be seen under the fluorescent microscope with light source at a specific wavelength. Due to the impracticality and various difficulties, these fluorescent fish species were not well received by consumers and did not achieve commercial success.

Chi-Yuan Chou et al. disclosed a DNA construct flanked at both ends by ITRs to increase the efficient expression of transgenic genes in medaka. A uniform transgene expression was achieved in the F0 and the following two generations (Chi-Yuan Chou et al., 2001, Transgenic Research 10: 303-315). Although a transgenic green fluorescence medaka has been described, method and condition of generating other transgenic fish with other fluorescent protein genes (such as red fluorescent protein) is different and cannot be easily deduced from the prior art because of the different strategies of genetic construction, gene expression, gene inheritance and uncertainties of the transgenic technique.

### SUMMARY OF THE INVENTION

The object of the invention is to use recombinant DNA techniques to establish a stable supply of fluorescence fish with desired transgenes.

Another object of the invention relates to a nucleic acid fragment comprising (1) an β-actin gene promoter of medaka; (2) a fluorescence gene; (3) inverted terminal repeats of adeno-associated virus; and (4) a basic part from pUC.

Another object of the invention relates to a plasmid comprising the nucleic acid fragment of the invention.

Yet another object of the invention relates to the method of generating a novel medaka that carry the fluorescent transgene and express fluorescent protein.

### BRIEF DESCRIPTION OF THE DRAWINGS

FIG. 1 illustrates the construction of plasmid pβ-DsRed2-1-ITR from pOBA-109 and pDsRed2-1-ITR.

FIG. 2 illustrates the process of generating transgenic medaka.

FIG. 3 is a photographic representation of a three-month-old transgenic medaka from F2 generation that were derived from founders that are successfully transfected with the nucleic acid fragment of the invention, pβ-DsRed2-1-ITR, demonstrating its red fluorescence expression.

### DETAILED DESCRIPTION OF THE INVENTION

To overcome disadvantages of transgenic fluorescent fish in the prior art, the current invention is of thorough and careful design with conceptual breakthrough. A plasmid construct, pB-DsRed2-1ITR, could be generated by introducing the β-actin gene promoter of medaka into expression vector pDsRed2-1-ITR (Clontech). The appropriate amount of the resulting plasmid, pβ-DsRed2-1-ITR, is then microinjected into the cytoplasm of fertilized eggs of medaka prior to the first cleavage. These eggs are screened to find progeny expressing fluorescence throughout their systemic skeletal muscle. Progeny with fluorescent transgene are then used for future breeding.

The term “medaka” in the invention is not limited but to that from Adrianichthyidae (Ricefishes) such as Oryzias javanicus, Oryzias latipes, Oryzias nigrimas, Oryzias luzonensis, Oryzias profundicola, Oryzias matanensis, Oryzias mekongensis, Oryzias minutillus, Oryzias melastigma, O. curvinotus, O. celebensis, X. oophorus, and X. saracinorum. The preferred medaka is not limited but to that from Oryziinae such as Oryzias javanicus, Oryzias latipes, Oryzias nigrimas, Oryzias luzonensis, Oryzias profundicola, Oryzias matanensis, Oryzias mekongensis, Oryzias minutillus, Oryzias melastigma, O. curvinotus, O. celebensis. The most preferred medaka is Oryzias latipes.

The present invention provides a gene fragment comprising (1) a β-actin gene promoter of medaka; (2) a fluorescence gene; (3) inverted terminal repeats (ITR) of adeno-associated virus; and (4) a basic part from pUC.

The preferred fragment of the invention is

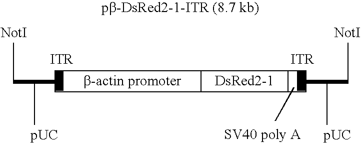

The present invention also provides a plasmid comprising the gene fragment of the invention.

The red fluorescent gene can be purchased from BD Bioscience Clontech. In the embodiment of the invention, pDsRed2-1 is used as the source of the red fluorescent gene. pDsRed2-1 encodes DsRed2, a DsRed variant engineered for faster maturation and lower non-specific aggregation. DsRed2 contains a series of silent base-pair changes that correspond to human codon-usage preferences for high expression in mammalian cells. In mammalian cell cultures when DsRed2 is expressed constitutively, red-emitting cells can be detected by fluorescence microscopy within 24 hours of transfection. Large insoluble aggregates of protein, often observed in bacterial and mammalian cell systems expressing DsRed1, are dramatically reduced in cells expressing DsRed2. The faster-maturing, more soluble red fluorescent protein is also well tolerated by host cells; mammalian cell cultures transfected with DsRed2 show no obvious signs of reduced viability—in those cell lines tested, cells expressing DsRed2 display the same morphology (e.g., adherence, light-refraction) and growth characteristics as non-transfected controls. pDsRed2-1 is a promoterless DsRed2 vector that can be used to monitor transcription from different promoters and promoter/enhancer combinations inserted into the multiple cloning site (MCS).

The method of the invention provides five improvements over other methods currently available:
1. The main body of the nucleic acid fragment of the invention is plasmid constructs such as pDsRed2-1-ITR, which are commercially available at an accessible price
2. The nucleic acid fragment of the invention enables the medaka to emit fluorescence throughout its systemic skeletal muscle.
3. The method of the invention, which comprises microinjecting the transgene construct into fertilized eggs, ensures the transgenic medaka emits fluorescence at its systemic skeletal muscle at a higher ratio with better quality.
4. The heterologous transgenic fish stably passes the transgene to the next generation. Thus natural breeding could be used to maintain the transgenic population and reduces the breeding cost.
5. The fluorescence of the transgenic medaka, which is emitted at its systemic skeletal muscle, can be easily seen by naked eyes. The red fluorescence is further intensified under light source of shorter wavelength, providing a higher entertainment value to ornamental fish.

Given above, the present invention provides a method of producing transgenic medaka with systemic fluorescence comprising:

(a) hatching the selected eggs to mature and cross with wild-type; and

(b) screening the progeny containing transgene and produce medaka with systemic fluorescence.

(c) constructing a plasmid including ITR, CMV promotor, a fluorescent gene, S40 poly A and ITR;

(d) replacing the CMV promoter with an β-actin gene promoter of medaka to produce a new plasmid construct;

(e) linearizing the new plasmid construct with NotI;

(f) microinjecting the appropriate amount of linearized plasmid construct into fertilized eggs of medaka;

(g) selecting the eggs with fluorescence;

(h) hatching the selected eggs to mature and crossing with wild-type; and

(i) screening the progeny containing transgene and produce medaka with systemic fluorescence.

Accordingly, the preferred linearized construct is selected from

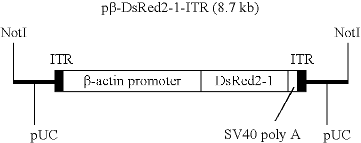

The preferred fluorescent gene used in the method of the invention is red fluorescent gene from pDsRed2-1.

In the method of producing transgenic medaka of the invention, the appropriate amount of NotI-linearized plasmid construct injected into the fertilized eggs is sufficient to introduce transgene into germ cell of medaka. The preferred amount of linearized plasmid construct injected into the fertilized eggs is 1-10 nl. The most preferred amount of linearized plasmid construct injected into the fertilized eggs is 2-3 nl.

The present invention also provides the transgenic medaka with systemic fluorescence produced from the method of the invention. The preferred medaka has systemic red fluorescence. Other color fluorescent fish may be generated by the same technique as blue fluorescent protein (BFP) gene, yellow fluorescent protein (YFP) gene and cyan fluorescent protein (CFP) gene.

### EXAMPLES

The examples below are non-limiting and are merely representative of various aspects and features of the present invention.

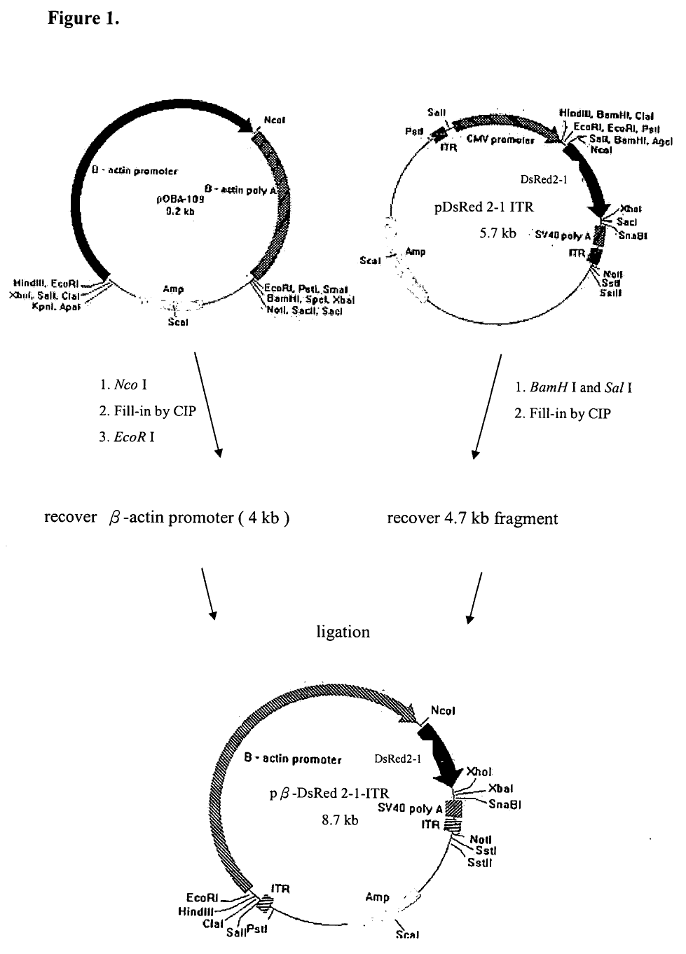

The method for producing medaka with red fluorescence:
1. Commercially available plasmid construct, pDsRed2-1 (Clontech) was used to generate the expression vector.
2. The DsRed fragment was from plasmid pDsRED2-1. The CMV promoter and two adeno-associated virus inverted terminal repeats (ITR) were ligated to the DsRed fragment as depicted in FIG. 1 to produce plasmid construct pDsRed2-1-ITR. The plasmid construct pDsRed2-1-ITR has shown higher expression stability.
3. Generating the novel plasmid construct: pβ-DsRed2-1-ITR

As illustrated in FIG. 1, the medaka β-actin gene promoter was obtained by digesting plasmid construct pOBA-109 with restriction enzymes NcoI and EcoRI. NcoI was used first, ends were filled in, and a subsequent digestion with EcoRI provided a 4 kb fragment.

As illustrated in FIG. 1, the CMV promoter was cut out by digesting the construct pDsRed2-1-ITR with restriction enzymes BamHI and SalI. Digestion with BamHI and SalI provided a 4.7 kb fragment. Then, the β-actin gene promoter of medaka was inserted into the plasmid construct, pDsRed2-1-ITR, at the position where the CMV promoter was cut out. The resulting plasmid construct had two 145 bp adeno-associated virus inverted terminal repeats (ITR). One ITR was located at the 3′ end of SV40 poly A. The other was located at the 5′ end of the β-actin gene promoter.

As illustrated in FIG. 1, the resulting plasmid construct, pβ-DsRed2-1-ITR, had a total length of 8.7 kb. pβ-DsRed2-1-ITR contained (1) the medaka β-actin gene promoter (for ubiquitous expression of whole body); (2) sea coral red fluorescent protein; (3) adeno-associated virus inverted terminal repeats; and (4) pUC plasmid construct basis.

The plasmid construct pβ-DsRed2-1-ITR was transformed into Escherichia coli 5a.
4. Linearization of the plasmid construct:

As illustrated in FIG. 1, appropriate amount of pβ-DsRed2-1-ITR was digested with proportional amount of Not I restriction enzyme. A small fraction of the digested product was analyzed by agarose gel electrophoresis to verify its linearity. The fragment length was 8.7 kb as expected. Then, the digested DNA products were extracted by a solution containing phenol:chloroform (1:1), precipitated by ethanol, air dried, then dissolved in PBS at a density of 10 μg/ml, which will be used for later cytoplasmic microinjection.
5. Cytoplasmic microinjection

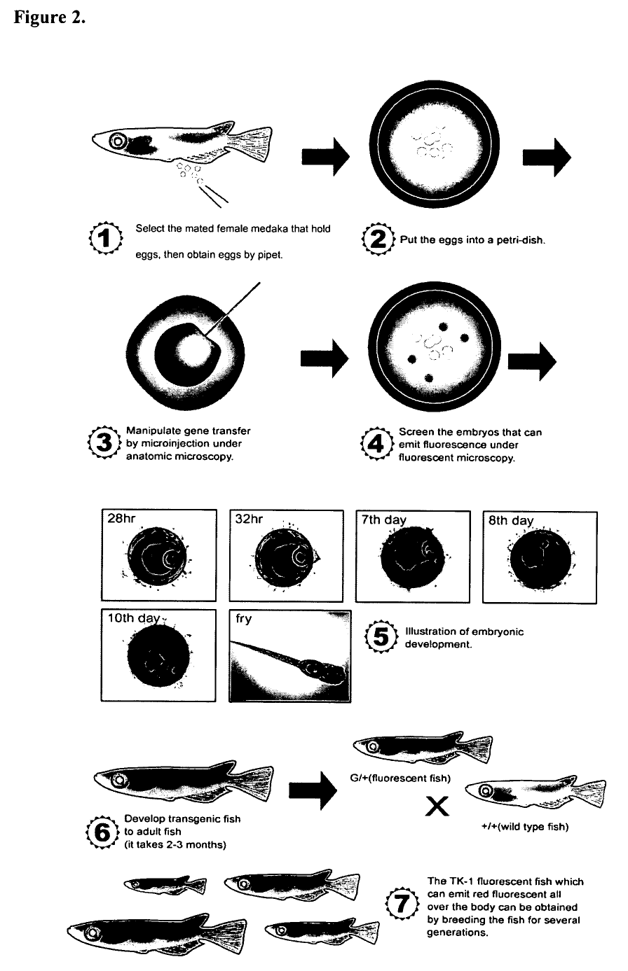

Collecting fertilized eggs: At 11 pm of the night before microinjection, and before the incubator entered the dark cycle, fish were collected and separated by separation net. On the next morning after the light cycle has begun, fish eggs were collected every 15-20 minutes as depicted in FIG. 2 step 1. In each microinjection session, 30-40 eggs were injected; 250-300 eggs were injected in each experiment as shown in FIG. 2 step 3.

Microinjection: The linearized construct was quantified and dissolved in 5×PBS with phenol red at the desired concentration. DNA was picked up by micro-capillary of medaka microinjector (Drummond) wherein the injection needle width of the micro-capillary was ranged 2-10 μm. As micro-needle enters the cell cytoplasm, the DNA injected was approximated 2-3 nl.

Hatching fertilized eggs: Injected eggs were rinsed with sterilized solution, cultured in incubator wherein the temperature was 26° C. The fluorescence could be observed in the developing embryo after 24 hours as illustrated in FIG. 2 step 4.
6. Fluorescent microscopy observation:

The injected embryo was placed in a dish with water. The distribution and intensity of the red fluorescence is observed under fluorescence microscope (Leica MZ-12; Fluorescence System: light source Hg 100 W; main emission wavelength 558 nm, and main absorption wavelength 583 nm, filter set RFP-Plus; photography system MPS60).
7. Germ-line transmission of transgene:

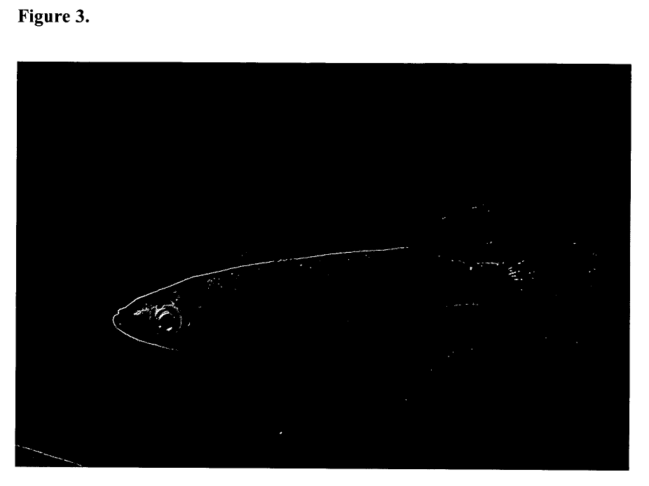

As showed in FIG. 2, red fluorescent medaka originated from embryos microinjected with pβ-DsRed2-1-ITR fragment were mated with wild type, to get the progeny that exhibited uniform fluorescence. The F1 with fluorescence expression was again mated with wild type to obtain the F2 progeny (FIG. 3), which all exhibited red fluorescent expression, and can be readily distinguished from fish without fluorescence expression. The difference between transgenic medaka and wild type could be better discerned under blue light.

The DNA fragment of the invention could be modified to carry other fluorescent genes, and thus fish with different fluorescence could be produced.

Other transgene construct comprising other fluorescence genes may be introduced to medaka eggs along with red fluorescence to make fish with various body colors.

The medaka of the invention can be broadly used in medicine research and researches in other fields of life sciences, for example, cell fusions, cloning, nuclear transfer, cell motility, cell targeting, and embryonic development research.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The embryos, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

What is claimed is:

1. A method of producing medaka with systemic fluorescence comprising:
(a) constructing a plasmid including ITR, CMV promotor, a fluorescent gene, S40 poly A and ITR;
(b) replacing the CMV promoter with an β-actin gene promoter of medaka to produce a new plasmid construct;
(c) linearizing the new plasmid construct with NotI;
(d) microinjecting the appropriate amount of linearized plasmid construct into fertilized eggs of medaka;
(e) selecting the eggs with fluorescence;
(f) hatching the selected eggs to mature and crossing with wild-type; and
(g) screening the progeny containing transgene and produce medaka with systemic fluorescence.

2. The method of claim 1, wherein the linearized plasmid is

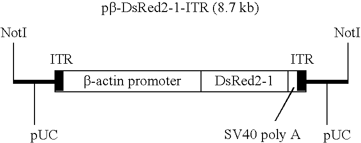

3. The method of claim 1, wherein the fluorescent gene is red fluorescent gene from pDsRed2-1.

4. The method of claim 1, wherein the appropriate amount of NotI-linearized plasmid construct injected into the fertilized eggs is sufficient to introduce transgene into germ cell of medaka.

5. The method of claim 4, wherein the appropriate amount of linearized plasmid construct injected into the fertilized eggs is 2-3 nl.

6. The method of claim 1, wherein the fluorescent gene is blue fluorescent protein (BFP) gene, yellow fluorescent protein (YFP) gene or cyan fluorescent protein (CFP) gene.

7. A medaka with systemic fluorescence produced from the method of claim 1.

8. The medaka of claim 7 that has systemic red fluorescence.

9. The medaka of claim 7 wherein the medaka is from Adrianichthyidae.

10. The medaka of claim 7 wherein the medaka is Oryzias javanicus, Oryzias latipes, Oryzias nigrimas, Oryzias luzonensis, Oryzias profundicola, Oryzias matanensis, Oryzias mekongensis, Oryzias minutillus, Oryzias melastigma, O. curvinotus, O. celebensis, X. oophorus, or X. saracinorum.

11. The medaka of claim 10 wherein the medaka is Oryzias javanicus, Oryzias latipes, Oryzias nigrimas, Oryzias luzonensis, Oryzias profundicola, Oryzias matanensis, Oryzias mekongensis, Oryzias minutillus, Oryzias melastigma, O. curvinotus or O. celebensis.

12. The medaka of claim 11 wherein the medaka is Oryzias latipes.

13. The medaka of claim 7 that has systemic blue fluorescence.

14. The medaka of claim 7 that has systemic yellow fluorescence.

15. The medaka of claim 7 that has systemic cyan fluorescence.
